SEQUENCE LISTING (46 sequences)

SEQ ID NO: 1 - DNA, 20 bp, synthetic construct
  misc_feature 1..20: note = Description of sequence: Primer, Name: MINK1_C2DeDif_F
  source 1..20: mol_type = other DNA
ccctctgctcccttctctct

SEQ ID NO: 2 - DNA, 20 bp, synthetic construct
  misc_feature 1..20: note = Description of sequence: Primer, Name: MINK1_C2DeDif_R
  source 1..20: mol_type = other DNA
aaaggttctccaggtgatgg

SEQ ID NO: 3 - DNA, 18 bp, synthetic construct
  misc_feature 1..18: note = Description of sequence: Primer, Name: PPP2R3A_C2DeDif_F
  source 1..18: mol_type = other DNA
ggccctcccacttttcag

SEQ ID NO: 4 - DNA, 20 bp, synthetic construct
  misc_feature 1..20: note = Description of sequence: Primer, Name: PPP2R3A_C2DeDif_R
  source 1..20: mol_type = other DNA
ggaaggaaaagaaagcctca

SEQ ID NO: 5 - DNA, 20 bp, synthetic construct
  misc_feature 1..20: note = Description of sequence: Primer, Name: MS4A5_C2DeDif_F
  source 1..20: mol_type = other DNA
gcctgagagattttgcgttc

SEQ ID NO: 6 - DNA, 20 bp, synthetic construct
  misc_feature 1..20: note = Description of sequence: Primer, Name: MS4A5_C2DeDif_R
  source 1..20: mol_type = other DNA
gagctgaaattgcaccactg

SEQ ID NO: 7 - DNA, 20 bp, synthetic construct
  misc_feature 1..20: note = Description of sequence: Primer, Name: BPNT1_C2DeDif_F
  source 1..20: mol_type = other DNA
gctctccgtaggtgcaagtc

SEQ ID NO: 8 - DNA, 20 bp, synthetic construct
  misc_feature 1..20: note = Description of sequence: Primer, Name: BPNT1_C2DeDif_R
  source 1..20: mol_type = other DNA
gctggaagacagctgaaacc

SEQ ID NO: 9 - DNA, 20 bp, synthetic construct
  misc_feature 1..20: note = Description of sequence: Primer, Name: LINC_C2DeDif_F
  source 1..20: mol_type = other DNA
ccctttctggccatagtgac

SEQ ID NO: 10 - DNA, 20 bp, synthetic construct
  misc_feature 1..20: note = Description of sequence: Primer, Name: LINC_C2DeDif_R
  source 1..20: mol_type = other DNA
aatgcagaaggcaagtctcc

SEQ ID NO: 11 - DNA, 20 bp, synthetic construct
  misc_feature 1..20: note = Description of sequence: Primer, Name: MARCHF2_C2DeDif_F
  source 1..20: mol_type = other DNA
aactgctaagtggccaatgc

SEQ ID NO: 12 - DNA, 20 bp, synthetic construct
  misc_feature 1..20: note = Description of sequence: Primer, Name: MARCHF2_C2DeDif_R
  source 1..20: mol_type = other DNA
ttgactaccccgttttgagc

SEQ ID NO: 13 - DNA, 20 bp, synthetic construct
  misc_feature 1..20: note = Description of sequence: Primer, Name: COG2_C2DeC_F
  source 1..20: mol_type = other DNA
ctgtggggacagaggctaag

SEQ ID NO: 14 - DNA, 20 bp, synthetic construct
  misc_feature 1..20: note = Description of sequence: Primer, Name: COG2_C2DeC_R
  source 1..20: mol_type = other DNA
ccctgagactgctctgcttc

SEQ ID NO: 15 - DNA, 20 bp, synthetic construct
  misc_feature 1..20: note = Description of sequence: Primer, Name: SUPT4H1_C2DeC_F
  source 1..20: mol_type = other DNA
ggctccaacgtaagttcacc

SEQ ID NO: 16 - DNA, 20 bp, synthetic construct
  misc_feature 1..20: note = Description of sequence: Primer, Name: SUPT4H1_C2DeC_F
  source 1..20: mol_type = other DNA
gggctgtgggacagtaaaga

SEQ ID NO: 17 - DNA, 22 bp, synthetic construct
  misc_feature 1..22: note = Description of sequence: Primer, Name: AP1G2_C2CuDif_F
  source 1..22: mol_type = other DNA
tttcctgcaagacttggtgttg

SEQ ID NO: 18 - DNA, 21 bp, synthetic construct
  misc_feature 1..21: note = Description of sequence: Primer, Name: AP1G2_C2CuDif_R
  source 1..21: mol_type = other DNA
cccaccacacataccctcttc

SEQ ID NO: 19 - DNA, 20 bp, synthetic construct
  misc_feature 1..20: note = Description of sequence: Primer, Name: HSD3B7_C2CuDif_F
  source 1..20: mol_type = other DNA
tcacactgtgggatgaggtc

SEQ ID NO: 20 - DNA, 20 bp, synthetic construct
  misc_feature 1..20: note = Description of sequence: Primer, Name: HSD3B7_C2CuDif_R
  source 1..20: mol_type = other DNA
cccattccaggaacagagac

SEQ ID NO: 21 - DNA, 20 bp, synthetic construct
  misc_feature 1..20: note = Description of sequence: Primer, Name: NYNRIN_C2CuDif_F
  source 1..20: mol_type = other DNA
ggctccccaccctactctac

SEQ ID NO: 22 - DNA, 20 bp, synthetic construct
  misc_feature 1..20: note = Description of sequence: Primer, Name: NYNRIN_C2CuDif_R
  source 1..20: mol_type = other DNA
tcccctcttgctagtggaag

SEQ ID NO: 23 - DNA, 20 bp, synthetic construct
  misc_feature 1..20: note = Description of sequence: Primer, Name: PRR7_C2CuDif_F
  source 1..20: mol_type = other DNA
ggaggagaatggcctctgac

SEQ ID NO: 24 - DNA, 20 bp, synthetic construct
  misc_feature 1..20: note = Description of sequence: Primer, Name: PRR7_C2CuDif_R
  source 1..20: mol_type = other DNA
gccagtcacctctaggatgc

SEQ ID NO: 25 - DNA, 20 bp, synthetic construct
  misc_feature 1..20: note = Description of sequence: Primer, Name: SATB1_C2CuDif_F
  source 1..20: mol_type = other DNA
ccctctacttcctgcacacc

SEQ ID NO: 26 - DNA, 20 bp, synthetic construct
  misc_feature 1..20: note = Description of sequence: Primer, Name: SATB1_C2CuDif_R
  source 1..20: mol_type = other DNA
gagcctgcttattgttgcac

SEQ ID NO: 27 - DNA, 20 bp, synthetic construct
  misc_feature 1..20: note = Description of sequence: Primer, Name: SLC13A2_C2CuDif_F
  source 1..20: mol_type = other DNA
cagcctccaagaacagaagg

SEQ ID NO: 28 - DNA, 20 bp, synthetic construct
  misc_feature 1..20: note = Description of sequence: Primer, Name: SLC13A2_C2CuDif_R
  source 1..20: mol_type = other DNA
acccagtctggcaatcagag

SEQ ID NO: 29 - DNA, 18 bp, synthetic construct
  misc_feature 1..18: note = Description of sequence: Primer, Name: SLC35D2_C2CuDif_F
  source 1..18: mol_type = other DNA
cggcaggaacaggaagag

SEQ ID NO: 30 - DNA, 21 bp, synthetic construct
  misc_feature 1..21: note = Description of sequence: Primer, Name: SLC35D2_C2CuDif_R
  source 1..21: mol_type = other DNA
aagcaaaacaagccctgagac

SEQ ID NO: 31 - DNA, 20 bp, synthetic construct
  misc_feature 1..20: note = Description of sequence: Primer, Name: UBOX5_C2CuDif_F
  source 1..20: mol_type = other DNA
ctcaagctggagagctacgg

SEQ ID NO: 32 - DNA, 20 bp, synthetic construct
  misc_feature 1..20: note = Description of sequence: Primer, Name: UBOX5_C2CuDif_R
  source 1..20: mol_type = other DNA
ggaagagaggttggcatcag

SEQ ID NO: 33 - DNA, 20 bp, synthetic construct
  misc_feature 1..20: note = Description of sequence: Primer, Name: CCDC144A_C2CuC_F
  source 1..20: mol_type = other DNA
gctgcagatgcagtgaagtc

SEQ ID NO: 34 - DNA, 18 bp, synthetic construct
  misc_feature 1..18: note = Description of sequence: Primer, Name: CCDC144A_C2CuC_R
  source 1..18: mol_type = other DNA
gatcactgccacgccaag

SEQ ID NO: 35 - DNA, 20 bp, synthetic construct
  misc_feature 1..20: note = Description of sequence: Primer, Name: PTCHD3_C2CuC_F
  source 1..20: mol_type = other DNA
caaggaacgcttcttcaagg

SEQ ID NO: 36 - DNA, 20 bp, synthetic construct
  misc_feature 1..20: note = Description of sequence: Primer, Name: PTCHD3_C2CuC_R
  source 1..20: mol_type = other DNA
ctgggtgaatgccaagaaag

SEQ ID NO: 37 - DNA, 20 bp, synthetic construct
  misc_feature 1..20: note = Description of sequence: Primer, Name: MIR4314_C2CuC_F
  source 1..20: mol_type = other DNA
agtgagaaagctggcctgtc

SEQ ID NO: 38 - DNA, 20 bp, synthetic construct
  misc_feature 1..20: note = Description of sequence: Primer, Name: MIR4314_C2CuC_R
  source 1..20: mol_type = other DNA
cagatccccttctcctaccc

SEQ ID NO: 39 - DNA, 19 bp, synthetic construct
  misc_feature 1..19: note = Description of sequence: Primer, Name: STAG2_C2CuC_F
  source 1..19: mol_type = other DNA
gaccgccactttcaaaacc

SEQ ID NO: 40 - DNA, 20 bp, synthetic construct
  misc_feature 1..20: note = Description of sequence: Primer, Name: STAG2_C2CuC_R
  source 1..20: mol_type = other DNA
tacacagaggcagcaacgag

SEQ ID NO: 41 - DNA, 20 bp, synthetic construct
  misc_feature 1..20: note = Description of sequence: Primer, Name: RXFP3_C1CuDif_F
  source 1..20: mol_type = other DNA
gccaccgtaactggtgagag

SEQ ID NO: 42 - DNA, 20 bp, synthetic construct
  misc_feature 1..20: note = Description of sequence: Primer, Name: RXFP3_C1CuDif_R
  source 1..20: mol_type = other DNA
gtttcctctgggactgcttg

SEQ ID NO: 43 - DNA, 20 bp, synthetic construct
  misc_feature 1..20: note = Description of sequence: Primer, Name: TIPARP_C1CuDif_F
  source 1..20: mol_type = other DNA
aggaggctggacaggagttg

SEQ ID NO: 44 - DNA, 18 bp, synthetic construct
  misc_feature 1..18: note = Description of sequence: Primer, Name: TIPARP_C1CuDif_R
  source 1..18: mol_type = other DNA
agtgacgcctcctgttgg

SEQ ID NO: 45 - DNA, 20 bp, synthetic construct
  misc_feature 1..20: note = Description of sequence: Primer, Name: HAUS4_C1CuDif_F
  source 1..20: mol_type = other DNA
gactgggaaacaggatggtc

SEQ ID NO: 46 - DNA, 20 bp, synthetic construct
  misc_feature 1..20: note = Description of sequence: Primer, Name: HAUS4_C1CuDif_R
  source 1..20: mol_type = other DNA
gcaaaatgcttggcgtagtg

(19) United States
(12) Patent Application Publication
(10) Pub. No.: US 2025/0230486 A1
(43) Pub. Date: Jul. 17, 2025

(54) METHODS AND RELATED MATERIALS FOR DETECTING DIFFERENTIATION POTENTIAL OF A CELL

(71) Applicant: The Florida State University Research Foundation, Inc., Tallahassee, FL (US)

(72) Inventor: Jonathan Dennis, Tallahassee, FL (US)

(21) Appl. No.: 18/999,577

(22) Filed: Dec. 23, 2024

Related U.S. Application Data
(60) Provisional application No. 63/613,355, filed on Dec. 21, 2023.

(51) Int. Cl. C12Q 1/6809; C12Q 1/34; C12Q 1/6806; C12Q 1/6851; C12Q 1/6886

(52) U.S. Cl. CPC C12Q 1/6809; C12Q 1/34; C12Q 1/6806; C12Q 1/6851; C12Q 1/6886; C12Q 2600/106; G01N 2333/916

(57) ABSTRACT

Provided are methods and materials for identifying differentiation potential of a sample comprising nucleosomes, including assays for highly sensitive and specific detection of the pathological potential of cells.

### CROSS-REFERENCE TO RELATED APPLICATIONS

This application claims the benefit of priority to U.S. Provisional Application No. 63/613,355, filed Dec. 21, 2023, which is incorporated by reference herein in its entirety.

### REFERENCE TO SEQUENCE LISTING

The sequence listing submitted on Dec. 23, 2024, as an .XML file entitled “10850-083US1_ST26.xml” created on Dec. 22, 2024, and having a file size of 60,887 bytes is hereby incorporated by reference pursuant to 37 C.F.R. § 1.52(e)(5).

### BACKGROUND OF THE INVENTION

The International Agency for Research on Cancer reported 19.3 million cancer cases and 10 million cancer deaths in 2020. Cancer ranks as the first or second cause of death before the age of 70 in the majority of the 195 countries on Earth. In addition to the sickness and death, costs reflecting treatment paid by patients, insurers, and patient time costs demonstrate the enormous economic burdens of cancer. The key to eliminating the personal and financial costs of cancer lies in the early detection of the disease. Early diagnosis relies on detecting the potentially cancerous tissue as early in disease progression as possible. Early detection is a critical public health strategy because it improves disease outcomes, increases survival, reduces complications associated with therapies, and reduces economic burden.

Early diagnosis presents a substantial challenge. There are few known early markers of cancer progression and no known markers of cancer potential. For example, colorectal cancer generally results from the oncogenic transformation of precancerous polyps in the colon or rectum. Polyps are surgically removed, and the patient is followed with a colonoscopy at regular intervals for 3-5 years. If cancer develops within that timeframe, the polyps are classified as “cancer adjacent polyps” (CAPs), and if no cancer develops, the polyps are classified as “cancer-free polyps” (CFPs). This post-hoc classification is frustrating for patients and physicians. Efforts to identify biomarkers to classify the potential of polyps as CAPs or CFPs in a manner contemporaneous with the polypectomy have failed.

The traditional measures of cell biology, such as gene expression, DNA methylation, protein modification, metabolite abundance, and histopathology, are powerful tools for characterizing the current state of a cell but have been historically poor predictors of what the cell is capable of in the future.

Chromatin sensitivity to micrococcal nuclease digestion is a novel genomic measurement and provides a unique readout compared to other measures of genome accessibility, such as ATAC-seq. Specifically, nucleosomes protect a population of genomic regions under light-digestion conditions; however, these footprints are lost in more heavily digested states (FIG. 1). Thus, chromatin regions in the genome display a biochemical property rendering them more susceptible to digestion.

### SUMMARY OF THE INVENTION

The present invention provides methods to determine differentiation potential of a cell, the method comprising:
a) exposing a sample comprising nucleosomes to Micrococcal nuclease (MNase) under conditions where MNase can digest nucleic acid, thereby producing digested nucleic acid;
b) detecting the digested nucleic acid;
c) comparing the digested nucleic acid of the sample to a standard, and
d) determining differentiation potential of the cell.

Also provided are such methods, wherein the differentiation potential is selected from the group comprising: cancer differentiation potential, pre-cancer differentiation potential, and/or developmental differentiation potential.

Also provided are such methods, wherein the sample comprising nucleosomes comprises whole cells, cell components, and/or cell extracts.

Also provided are such methods, wherein the digested nucleic acid is detected by amplification.

Also provided are such methods, wherein the amplification is selected from the group comprising: ligase chain reaction (LCR), loop mediated isothermal amplification (LAMP), multiple displacement amplification, self-sustained sequence replication (3SR), rolling circle amplification (RCA), nucleic acid sequence-based amplification (NASBA), polymerase chain reaction (PCR), and quantitative polymerase chain reaction (qPCR).

Also provided are such methods, wherein the amplification utilizes primer pairs comprising the nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46, or primer pairs with 80% or more homology to these sequences.

Also provided are such methods, wherein differentiation potential is pathology potential.

Also provided are such methods, wherein the sample comprising nucleosomes is a pathology sample.

Also provided are such methods, wherein the digested nucleic acid is detected by qPCR.

Also provided are such methods, wherein the pathology sample is a colon polyp.

Also provided are such methods, wherein the amplification utilizes primer pairs comprising the nucleic acid sequences: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; or primer pairs with 80% or more homology to these sequences.

Also provided are such methods, wherein the pathology potential is cancer potential and/or an inflammatory potential.

Also provided are such methods, wherein detection is via primers that are capable of differentiating between tightly-coiled chromatin and loosely-coiled chromatin.

Also provided are such methods, wherein detection is via primers that are capable of identifying tightly-coiled chromatin.

Also provided are such methods, wherein the pathology potential of the cell is higher when the primers amplify a low number of DNA products.

Also provided are such methods, wherein the pathology potential of the cell is lower when the primers amplify a high number of DNA products.

Also provided are such methods, which have greater than 90% sensitivity and specificity.

Also provided are such methods, which provides pathology potential determination the same day as the pathology sample is removed from a patient.

The present invention provides methods of predicting, preventing, diagnosing, or treating a disease in a subject in need thereof, the method comprising determining the differentiation potential of a cell according to the methods described herein, and predicting, preventing, diagnosing, or treating a disease if the pathology potential is high.

The present invention provides methods of predicting, preventing, diagnosing, or treating a disease in a subject in need thereof, the method comprising determining the differentiation potential of a cell according to the methods described herein, and predicting, preventing, diagnosing, or treating a disease if the pathology potential is low.

The present invention provides kits comprising primers designed to amplify tightly-coiled chromatin in a nucleosome-containing sample and instructions to use in a qPCR test.

Also provided are such kits, which further comprise MNase.

Also provided are such kits, which further comprise control primers.

Also provided are such kits, wherein the primers are in pairs comprising nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46, or primer pairs with 80% or more homology to these sequences.

The invention provides Isolated primer pairs comprising nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46, or primer pairs with 80% or more homology to these sequences.

### BRIEF DESCRIPTION OF FIGURES

FIG. 1A shows MNase sensitivity. DNA extracted from crosslinked human cells digested with a titration of MNase. Lowest band is mononucleosomally protected DNA. FIG. 1B shows sensitive and resistant nucleosomes. Example loci displaying occupancy profiles of nucleosomes isolated under light and heavy digestion conditions. Example sensitive and resistant nucleosomes are highlighted in blue and red, respectively.

FIG. 2A shows that nucleosome distribution is largely invariant between CFP and CAP. Heatmaps displaying total mTSS-seq nucleosome distribution data for 2000 base pairs centered on the TSS of all human genes. Nucleosome distribution data for each patient polyp sample was organized by k-means clustering (k=7) using the sort order of the first heatmap. Average profiles for each cluster are shown below each individual heatmap. FIG. 2B shows that nucleosome sensitivity varies between CAP and CFP. Heatmaps same as above, except nucleosome sensitivity data for each patient polyp sample was calculated by computing the log 2 ratio of reads from the light MNase digest over the reads of the heavy MNase digest.

FIGS. 3A-E depict that nucleosome sensitivity at the +1 and −1 nucleosome positions can discriminate between CFP and CAP. Heatmaps showing nucleosome sensitivity at −1 nucleosome (50-250 bp upstream) (FIGS. 3A-3B) and +1 nucleosome (50-250 bp downstream) (FIGS. 3C-3D) show a distinctive pattern for CAP and CFP. Columns represent patient samples, rows are genes. Cells are the sensitivity value of the location are shown with yellow indicating sensitivity and blue indicating resistance to MNase.

FIG. 4 shows that qPCR can capture nuclease sensitivity differences between CFP and CAP. qPCR results for each of 26 replicate reactions for one CFP (blue) and one CAP (red) patient sample. The left box shows qPCR of heavily digested patient polyp samples and demonstrates reduced PCR efficiency at CFP regions identified as MNase sensitive. The center box shows qPCR of heavily digested patient polyp samples and demonstrates reduced PCR efficiency at CAP regions identified as MNase sensitive. The box on the right is a control showing qPCR of uncut genomic DNA using primer sets 1 and 2, demonstrating that MNase sensitivity is responsible for the differential amplification (delta Ct) of heavily digested samples. Data for each category is sorted by delta Ct values.

FIG. 5A shows a schematic of DNA tightly wrapped on a histone that cannot be cut (“RESISTANT”). FIG. 5B shows a schematic of DNA tightly wrapped on a histone that can be cut (“SENSITIVE”).

FIG. 6 shows that genome sensitivity can discriminate benign polyps that will become cancer and those that won't. Archived benign polyps were received from patients that did and did not develop cancer. Sensitivity was measured, then patients were blindly classified according to gene sensitivity. Two clear groups were identified. On the left side are resistant genes. The right side shows sensitive genes.

### DETAILED DESCRIPTION OF THE INVENTION

### Definitions

The term “gene” as used in this specification refers to a segment of deoxyribonucleotides (DNA) possessing the information required for synthesis of a functional biological product such as a protein or ribonucleic acid (RNA).

The term “genetic engineering” is used to indicate various methods involved in gene manipulation including isolation, joining, introducing of gene(s) as well as methods to isolate select organisms containing the manipulated gene(s).

As specified herein, the term “DNA construct” refers to a sequence of deoxyribonucleotides including deoxyribonucleotides obtained from one or more sources.

The term “gene expression” refers to efficient transcription and translation of genetic information contained in concerned genes.

The term “recombinant” cells or population of cells refers to cells or population of cells into which an exogenous nucleic acid sequence is introduced using a delivery vehicle such as a plasmid.

The term “microorganism” mentioned herein refers to one or more forms/species of bacteria or yeast.

The term “nucleic acid” as used herein means natural and synthetic DNA, RNA, oligonucleotides, oligonucleosides, and derivatives thereof. For ease of discussion, such nucleic acids are at times collectively referred to herein as “constructs,” “plasmids,” or “vectors.”

A “primer” is a nucleic acid that contains a sequence complementary to a region of a template nucleic acid strand and that primes the synthesis of a strand complementary to the template (or a portion thereof). Primers are typically, but need not be, relatively short, chemically synthesized oligonucleotides (typically, deoxyribonucleotides). In an amplification, e.g., a PCR amplification, a pair of primers typically define the 5′ ends of the two complementary strands of the nucleic acid target that is amplified.

As used herein, “complementary” or “complementarity” refers to the ability of a nucleotide in a polynucleotide molecule to form a base pair with another nucleotide in a second polynucleotide molecule. For example, the sequence 5′-A-C-T-3′ is complementary to the sequence 3′-T-G-A-5′. Complementarity may be partial, in which only some of the nucleotides match according to base pairing, or complete, where all the nucleotides match according to base pairing. For purposes of the present invention “substantially complementary” refers to 80% or greater identity over the length of the target base pair region. The complementarity can also be 50, 60, 70, 75, 80, 85, 90, 91, 92, 93, 94, 95, 96, 97, 98, 99, or 100% complementary, or any amount below or in between these amounts.

“Homology” and “homologous” refers to the state of having the same or similar relation, relative position, and/or structure. For purposes of the present invention “homology” refers to 80% or greater sequence identity over the length of the referenced primer. The complementarity can also be 50, 60, 70, 75, 80, 85, 90, 91, 92, 93, 94, 95, 96, 97, 98, 99, or 100% complementary, or any amount below or in between these amounts. Homology may be determined using sequence comparison programs such as GAP (Deveraux et al., 1984, Nucleic Acids Research 12, 387-395), which is incorporated herein by reference.

“Micrococcal nuclease” (EC 3.1.31.1), also referred to as spleen endonuclease, thermonuclease, nuclease T, micrococcal endonuclease, nuclease T′, staphylococcal nuclease, spleen phosphodiesterase, Staphylococcus aureus nuclease, Staphylococcus aureus nuclease B, and ribonucleate (deoxynucleate) 3′-nucleotidohydrolase, is an endo-exonuclease that preferentially digests single-stranded nucleic acids. This can include the linker region between nucleosomes, which allows for the digestion of free DNA ends toward the core nucleosome.

A “nucleosome” is a basic unit of DNA chromatin in eukaryotic organisms. Eukaryotic chromatin is composed of DNA and DNA is present in the cell nucleus. A nucleosome consists of units that assist with the DNA packaging in eukaryotes. The nucleosome is made up of a DNA sequence of roughly 150 base pairs wrapped around a core of histone proteins.

A “histone” is a water-soluble alkaline protein. It is rich in lysine and arginine. There are five major families of histones: H1, H2A, H2B, H3, and H4. Their positive charges allow them to associate with DNA, which is negatively charged. Histones can function as spools for nucleic acid to wrap around.

By “cells” is meant any cell capable of differentiating and/or de-differentiating, including: erythrocytes, platelets, bone marrow cells, vascular endothelial cells, lymphocytes, hepatocytes, neuronal cells, glial cells, bronchial endothelial cells, epidermal cells, respiratory interstitial cells, adipocytes, dermal fibroblasts, muscle cells, and any cellular subtype or cell. Cells can have functions that are enhanced or disrupted by virtue of differentiation or de-differentiation, including: exocrine, barrier, hormone, autonomic, sensory, metabolic, secretory, contractile, immune, and structural. Human, livestock, companion animal, and research animal cells are contemplated herein.

As used herein, “amplify, amplifying, amplifies, amplified, amplification” refers to the creation of one or more identical or complementary copies of the target DNA. The copies may be single stranded or double stranded. Amplification can be part of a number of processes such as extension of a primer, reverse transcription, polymerase chain reaction, nucleic acid sequencing, rolling circle amplification and the like. For the present invention, amplification of nucleic acids may be according to any method, including, but not limited to ligase chain reaction (LCR), loop mediated isothermal amplification (LAMP), multiple displacement amplification, self-sustained sequence replication (3SR), rolling circle amplification (RCA), nucleic acid sequence-based amplification (NASBA), polymerase chain reaction (PCR), and quantitative polymerase chain reaction (qPCR).

As used herein, the term “derived from” generally refers to an origin or source, and may include naturally occurring, recombinant, unpurified or purified molecules. A nucleic acid derived from an original nucleic acid may comprise the original nucleic acid, in part or in whole, and may be a fragment or variant of the original nucleic acid. A nucleic acid derived from a biological sample may be purified from that sample.

As used herein, the term “diagnose” or “diagnosis” of a status or outcome generally refers to predicting or diagnosing the status or outcome, determining predisposition to a status or outcome, monitoring treatment of a subject (e.g., a patient), diagnosing a therapeutic response of a subject (e.g., a patient), and prognosis of status or outcome, progression, and response to particular treatment.

As used herein, the term “subject” generally refers to an individual, entity or a medium that has or is suspected of having testable or detectable genetic information or material. A subject can be a person, individual, or patient. The subject can be a vertebrate, such as, for example, a mammal. Non-limiting examples of mammals include humans, simians, farm animals, sport animals, rodents, and pets. The subject may be displaying a symptom(s) indicative of a health or physiological state or condition of the subject, such as a cancer or a stage of a cancer of the subject. As an alternative, the subject can be asymptomatic with respect to such health or physiological state or condition.

### General Description

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures can ultimately achieve the same result.

The invention is further illustrated below in conjunction with specific embodiments. It is to be understood that the examples are merely illustrative of the invention and are not intended to limit the scope of the invention. The experimental methods in which the specific conditions are not indicated in the following examples are usually carried out according to the conditions described in the conventional conditions, for example, Sambrook et al., Molecular Cloning: Laboratory Manual (New York: Cold Spring Harbor Laboratory Press, 1989) manufacturing conditions or according to the conditions recommended by the manufacturer. Unless otherwise stated, percentages and parts are by weight and parts by weight.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

### Methods of Determining Differentiation Potential

Disclosed herein is a method to determine differentiation potential of a cell, the method comprising: exposing a sample comprising nucleosomes to Micrococcal nuclease (MNase) under conditions where MNase can digest nucleic acid, thereby producing digested nucleic acid; detecting the digested nucleic acid; comparing the digested nucleic acid of the sample to a standard, and determining differentiation potential of the cell. This is also referred to herein as “nucleosome sensitivity assay.”

“Differentiation potential” includes the characteristics individual cells take on during the process of differentiation. For example, differentiation potential can refer to a switch by the cell from one pattern of gene expression to another, including gene pattern changes associated with age, health status, genomic influence, epigenetics, pathogens, or environmental cues. Differentiation potential includes cellular changes due to nucleic acid mutation that results in signaling, packaging, folding, or processing in either the nucleic acid(s) or amino acid(s), either in the context of a single event or a cascading series of events.

In some instances, a cell or cell component is transformed from within (e.g., failures of nucleic acid repair during cell division) and, in other instances, the cell(s) or cellular component(s) is transformed from without (e.g., by viruses or other vectors, cell membrane-influencing compositions or environments, and/or other mutagens, such as radiation). This can also happen to an immature cell as it goes through the differentiation process.

Differentiation potential can also refer to “de-differentiation,” such as occurs when cells grow in reverse, from a partially or terminally differentiated stage to a less differentiated stage within their own lineage. This can be the result of changes in the expression pattern of genes.

The differentiation potential can be determined by comparing the cells to a control, such as a cell which is considered “normal” or “standard.” It can also refer to cells taken from the same individual at a different time point.

One of skill in the art will appreciate that the standard can be compared to the test sample, and differences in nucleic acid digestion patterns of DNA associated with nucleosomes can be determined. These patterns can then be used to determine differentiation potential of various cells. These patterns can be determined by using algorithms, and can be done by computer or machine learning techniques.

For example, in the case of cancer, a cell which has been obtained from a subject can be processed according to the methods disclosed herein. The results can be compared to a standard, such as the results from normal, non-cancerous cells, or to a sample obtained from the subject at a different time. Disclosed herein are assays which make use of nucleosomes. These assays can be performed using purified nucleosomes, or can comprise whole cells, cell components, and/or cell extracts.

The digested DNA can be subjected to amplification, for example. The DNA can be detected through amplification/detection methods. For example, the amplification can be selected from the group comprising: ligase chain reaction (LCR), loop mediated isothermal amplification (LAMP), multiple displacement amplification, self-sustained sequence replication (3SR), rolling circle amplification (RCA), nucleic acid sequence-based amplification (NASBA), polymerase chain reaction (PCR), and quantitative polymerase chain reaction (qPCR).

Specifically contemplated herein are rapid qPCR assays to classify the cancer, inflammatory, or other pathological potential of resected tissue. Specifically provided are processes that include digestion of tissue samples with micrococcal nuclease and predicting, detecting, and preventing pathologies based on amplification of digested nucleic acid products via primer-probe sets for analysis by qPCR. The results of the qPCR on the digested material will allow for the classification of tissue samples, for example, benign colon polyps as cancer-free or cancer-associated.

As stated above, a major problem in cancer diagnostics is a dearth of assays that can detect the oncogenic potential of tissues for the earliest possible cancer detection. Nucleosome sensitivity to Micrococcal nuclease (MNase) is a powerful indicator of the transformation potential of colon polyps. Furthermore, the sensitivity assay identifies targets readily testable by quantitative PCR (qPCR). The biomarkers targets identified by sensitivity assay are readily translatable to existing qPCR platforms, like those developed for infectious disease testing (e.g., COVID-19, MRSA, Influenza, RSV, StrepA, etc.). The present invention provides the first same-day colonoscopy qPCR early diagnostic for colorectal carcinoma potential.

The value of this technology lies in its ability to detect the potential of an individual to develop pathologies at the earliest possible time. This is particularly important for colorectal cancer, as it has no symptoms in its early stages, and early detection can significantly improve patients' chances of survival. Regular screening tests, such as colonoscopies, are the best method of early detection. Colonoscopies identify and remove neoplastic polyps in the colon. Not all polyps are classified as cancerous or precancerous; most are classified as benign. While resection of a benign polyp during a colonoscopy significantly reduces the risk of developing colorectal cancer, there is still a chance that cancer can develop in the exact location or elsewhere in the colon or rectum. The present invention determines whether the resected benign polyp has the potential to become cancerous and will significantly positively impact patients, healthcare providers, and society at large. This earliest detection would improve patient outcomes and allow less aggressive therapies such as surgery or radiation therapy. From the healthcare provider's perspective, early detection results in less resource-intensive solutions for the healthcare system, lower hospitalization rates, and lower treatment costs. Early detection also has broader societal benefits, including increased survival rates and reduced productivity losses associated with treatment.

The invention also impacts clinical practice for other adenocarcinomas. This is important because adenocarcinomas are the most common type of cancer affecting organs, and predicting aggressiveness represents a real challenge. Current methods to characterize the aggressiveness of an adenocarcinoma (e.g., tumor size, grade, stage, and molecular features) involve a high degree of uncertainty. Nonetheless, these measurements are necessary to develop a diagnosis and treatment plan specific to the patient's needs and likelihood of success. The present methods help reduce that uncertainty in characterizing other adenocarcinomas. Methods included herein apply to detecting and predicting the cellular potential in lung adenocarcinoma, which accounts for 40% of all lung cancer; breast adenocarcinoma: the second most common cancer in women; prostate adenocarcinoma, the most common type of cancer in men; and pancreatic adenocarcinoma: a particularly aggressive cancer with a poor prognosis.

Further, the chromatin sensitivity-informed qPCR methods apply to other diseases. For example, differential inflammatory responses to pathogen exposure in an MNase sensitivity analysis of peripheral blood mononuclear cells (PBMCs).

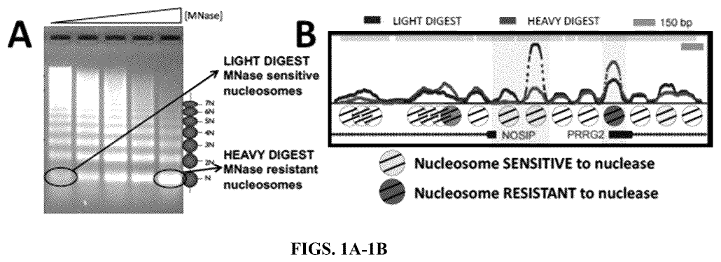

Chromatin sensitivity to micrococcal nuclease digestion is a novel genomic measurement and provides a unique readout compared to other measures of genome accessibility, such as ATAC-seq. Specifically, nucleosomes protect a population of genomic regions under light-digestion conditions; however, these footprints are lost in more heavily digested states (FIGS. 1A-1B). Thus, chromatin regions in the genome display a biochemical property rendering them more susceptible to digestion.

The invention uses a novel measurement of chromatin sensitivity to predict the oncogenic potential of neoplastic tissue classified as benign. The chromatin sensitivity assay identifies regions of the genome that are susceptible to nuclease digestion, hydrolysis of the phosphodiester linkage. In this invention, disclosed are regions of the genome that are differentially susceptible to digestion by MNase in cancer-free and cancer-associated benign polyps. For example, a region of the BPNT1 gene, a prognostic indicator for cancer, is more nuclease sensitive in cancer-associated and more nuclease resistant in cancer-free polyps. After digestion with MNase according to the present methods, the BPNT1 gene will be more likely to generate a PCR product from primers at this location in cancer-free polyps than in a cancer-associated polyp. (see Example 3. “Preliminary study 3”). This work is innovative because it leverages the fundamental principles of complex, labor-intensive, and expensive next-generation sequencing chromatin sensitivity assay to develop target primer-probe sets for a streamlined, rapid, and cost-effective qPCR diagnostic assay.

This disclosure, in certain aspects, is generally directed to methods for determining the differentiation state of a cell, based on a combination of biological detection methods and optionally, image analysis methods. Biological detection methods comprise techniques used to detect biological changes in a cell, and may further comprise taking measurements or acquiring images of a cell or cellular structure in a non-invasive, non-perturbing and non-destructive manner. Methods of the present invention are not limited to biological changes in a particular type of cell or cellular structure. In one aspect, specific biological changes may be related to the chromatin in the nucleus of a cell, and characteristics of the chromatin may be used to determine the differentiation state of the cell.

### Kits and Primers

The present invention also provides a kit comprising MNase as described in the present invention. In a preferred embodiment of the present invention, the kit further includes a container, an instruction, a buffer, and the like. The kit can also comprise specific primers, such as any combination of those disclosed in SEQ ID NOS: 1-46.

The invention also relates to primers which are capable of amplifying nucleic acid. This nucleic acid can be released by MNase, and can therefore be detected in a differential assay to determine differentiation potential of that cell. These primers can hybridize, for example, under stringent conditions. In the present invention, “stringent conditions” refers to: (1) hybridization and elution at lower ionic strength and higher temperature, such as 0.2×SSC, 0.1% SDS, 60° C.; or (2) additional denaturants during hybridization, such as 50% (v/v) formamide, 0.1% fetal bovine serum/0.1% Ficoll™, 42° C., etc.; or (3) hybridization occurs only under the identity between the two sequences at least over 90%, preferably over 95%. Also, polypeptides encoded by hybridizable polynucleotides have the same biological functions and activities as mature polypeptides.

In addition to SEQ ID NOS: 1-46, the present invention also contemplates primers with 80%, 81%, 82%, 83%, 84%, 85%, 86%, 87%, 88%, 89%, 90%, 91%, 92%, 93%, 94%, 95%, 96%, 97%, 98%, or 99% identity to any of SEQ ID NOS: 1-46.

### EXAMPLES

The early detection of the transformation of cells is critical for the effective treatment and cure. Herein described are a series of rapid qPCR tests for use in clinical settings.

### Example 1. Preliminary Study 1

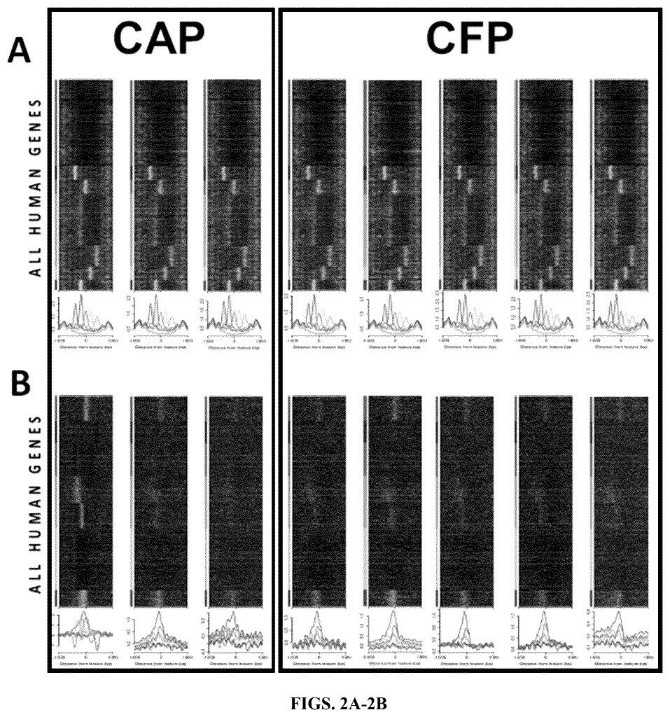

Chromatin sensitivity distinguishes benign colon polyps based on their transformation potential. MNase-Transcription Start Site Sequence Capture method (mTSS-seq) was used as established previously to produce high-resolution nucleosome occupancy (FIG. 2A) and nuclease sensitivity (FIG. 2B) maps of eight colorectal polyps. Nucleosome occupancy maps among patient samples are strikingly similar (FIG. 2A). Differences between CFP and CAP were tested to determine if they could be captured by measuring nucleosome sensitivity. To accomplish this, patient-derived polyps were digested with two concentrations of MNase, light and heavy (FIGS. 1A-1B). High-resolution heat maps were developed by calculating the log 2 ratio of the normalized reads mapped for 2 kb around TSSs in light over heavy MNase digests. Clear differences in MNase sensitivity are observed within and between CFP and CAP samples (FIG. 2B). It is notable that the sensitivity pattern is not maintained across samples. These results demonstrate that measures of nucleosome sensitivity will give insight into differences between CFP and CAP.

### Example 2. Preliminary Study 2

Chromatin sensitivity can identify differential sensitivity locations that distinguish CFP from CAP benign polyps.

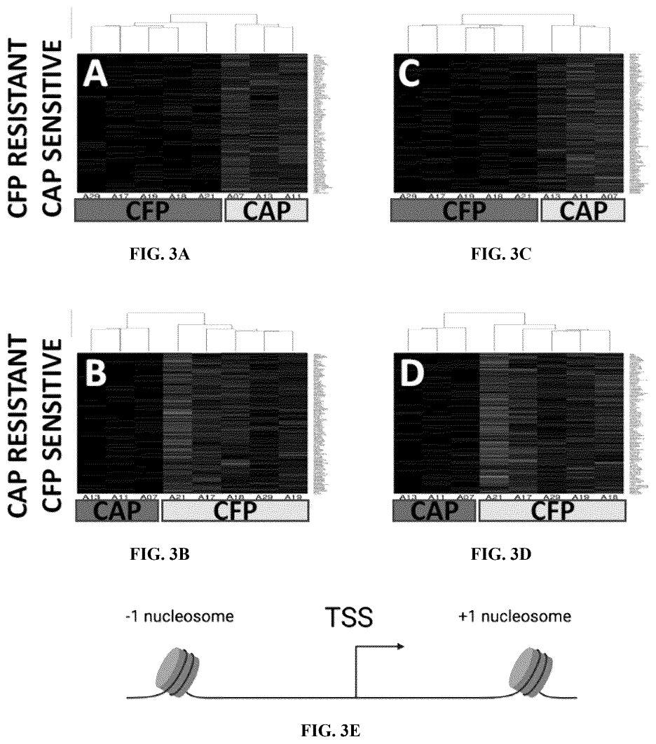

The prevailing view of promoter organization suggests that the strongly positioned +1 and −1 nucleosomes, most proximal to the TSS, establish the statistical positioning of nucleosomes at the promoter, and play important roles in gene regulation. To test the idea that the sensitivity of the −1 and +1 nucleosomes would give insights into differential promoter regulation between CFP and CAP. To accomplish this, data was filtered at the −1 nucleosome position (−50 to −250 bp relative to TSS) or the +1 nucleosome position (+50 to +250 bp relative to TSS). All −1 and +1 locations were identified in which all patient-derived samples had a log 2 ratio of normalized reads in light over heavy digests greater than or equal to 0.3 to be identified as sensitive or less than −0.3 to be identified as resistant. MNase Resistant Nucleosomes (MRN) and MNase Sensitive Nucleosomes (MSN) were identified. There were two possibilities for each location CFP MRN and CAP MSN, or CFP MSN and CAP MRN. At the −1 nucleosome position, 538 CFP MRN and CAP MSN (FIG. 3A) were identified as well as 644 CFP MSN and CAP MRN (FIG. 3B). At the +1 nucleosome position, 611 CFP MRN and CAP MSN (FIG. 3C) were identified as well as 687 CFP MSN and CAP MRN (FIG. 3D). These results further support the ideas underpinning the proposed work: that MNase sensitivity is an important analytical tool to identify functionally different promoter architectures that can discriminate CFP and CAP.

### Example 3. Preliminary Study 3

Quantitative PCR can be used to assay genomic sensitivity to MNase.

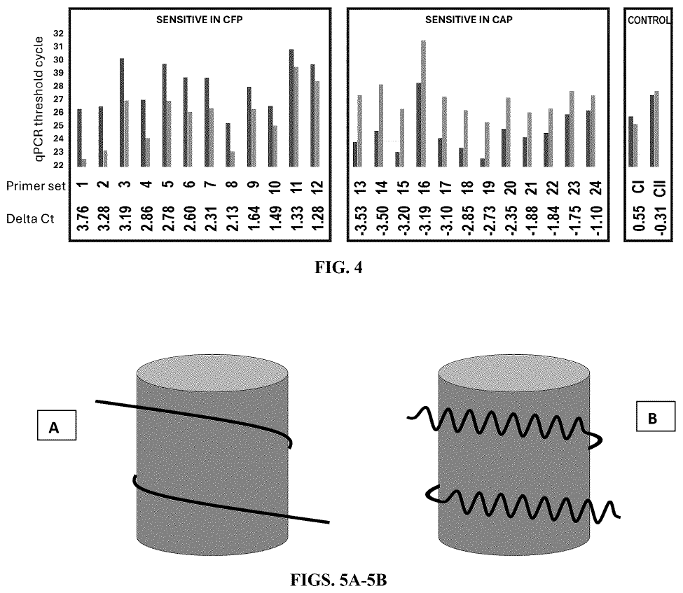

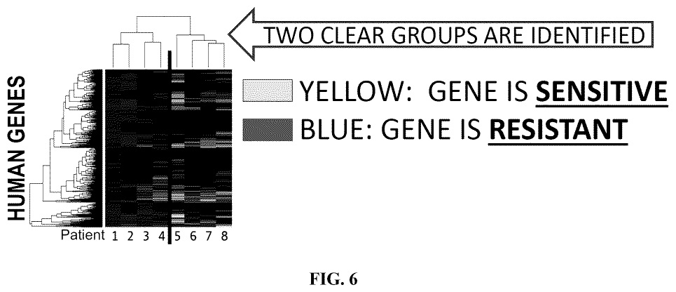

This study was to validate that regions identified as differentially sensitive to MNase by sequencing analysis could be used in a qPCR assay to distinguish CFP and CAP. Because the chromatin sensitivity assay reflects sensitivity to nuclease, areas identified as sensitive are cleaved by the nuclease rendering them poorer templates for PCR. Regions identified as resistant to nuclease will be less cleaved by the nuclease making them relatively better templates for PCR. Using the analysis presented in Example 2, Preliminary Study 2, six genomic locations for qPCR analysis of sensitivity were identified. QPCR primers for these regions were designed. Four loci were selected that demonstrated greater MNase sensitivity in CAP (MSNs) than CFP at the −1 nucleosome position (FIG. 3A). The studies were intended to identify whether regions identified as MNase sensitive would PCR amplify less efficiently than those identified as MNase resistant. Five 10 μm cryostat slices of patient polyps were heavily MNase-digested for 5 min at 37° C. using 200 unit/mL of MNase. DNA was isolated and DNA fragments were selected that were less than 500 bp (0.5×SPRIselect, Beckman Coulter) for qPCR analysis. Eight replicate qPCR CAP and CFP reactions for each primer set were prepared for the heavily digested samples. As a control, two qPCR reactions using total uncut genomic DNA from these samples were used. The qPCR amplifications were less efficient for the CAP-sensitive samples (FIG. 4). DCts of 3 to 5 cycles were observed for the CAP-sensitive samples. Total uncut genomic DNA showed no significant differences in Ct values (FIG. 4). These results demonstrate that the DCts for the heavily MNase-digested samples reflect bona fide differences in chromatin sensitivity between the CAP and CFP and support the approach of translating sensitivity measurements into an accurate, rapid, and cost-effective clinical qPCR diagnostic test.

### Example 4. Summary of Preliminary Studies

These studies accomplish three critical proof-of-principle milestones. In preliminary study 1, the results have shown that nucleosome sensitivity to nuclease, rather than nucleosome position or occupancy, is a more informative measure of chromatin dynamics in tissue. In preliminary study 2, the results have shown the ability to identify regions of differential sensitivity that distinguish apparently benign polyps free of cancer, CFP, from those that are associated with cancer, CAP. Finally, in preliminary study 3, qPCR as a rapid and cost-effective tool that accurately reflects the Illumina sequencing-based sensitivity work was validated. These preliminary studies demonstrate the present methods.

### Example 5. SA 1

Identify chromatin sensitivity-based biomarkers that distinguish cancer-free from cancer-associated resected patient polyps (See Primer Pair Table 1). The study identifies 48 differentially sensitive regions that distinguish patient CFP from patient CAP and prepare qPCR primers for these regions. Further, this study allows curation of a set of 12 primer sets (4 control and 8 diagnostic) able to differentiate CFP from CAP.

The study allows for robust MNase-sensitivity-based markers of transformation potential. Rigorous identification of these markers faces includes solving the issues of cellular heterogeneity, individual patient genetic background, the complex genetic architecture of the disease, and environmental factors. This study requires increased numbers of samples. This analysis is on par with 100,000 SNPs queried from 4 potential nucleosome positions (−2, −1, +1, and +2) of the 2000 bp surrounding the TSS of each of 20,000 human genes (80,000 nucleosome locations). Given that 5-10% of apparently benign polyps will become cancerous, 800 patient samples are analyzed and 50-100 of those samples are CAP, with the remaining being CFP.

Accio Biobank Online (biobankonline.com) receives benign polyp specimens that have been de-identified apart from gender and age of donor, and pathological classification. 48 samples per week are shipped on dry ice of >200 mg of treatment-naive, polyps flash-frozen within 2 hours of resection and classified as CFP or CAP. 50 mgs of formaldehyde crosslinked patient polyps are digested for 5 min at 37° C. with MNase at 20 units/mL (light) and 200 unit/mL of MNase (heavy). DNA is isolated and mononucleosmal fragments is gel purified. Illumina sequencing libraries are prepared from the mononucleosomal fragments and run on the Illumina NovaSeq 6000 to generate 50 million PE150 reads per sample. Sequencing is processed as described previously. Nucleosome distributions are normalized to fragments per million in the 2 kb surrounding each TSS. Sensitivity analysis (as in FIGS. 2A-2B and FIGS. 3A-3E) is performed in R using lab-developed software, GENMAT.

This invention discloses that different loci are differentially sensitive between CFP and CAP (FIGS. 3A-3E). Different polyp samples and even different locations within the same polyp exhibit cellular heterogeneity. Pathological classification of each sample aids in the interpretation of sensitivity maps. Additionally, the genetic background of the individual is considered in the sensitivity analysis. Monitoring the genetic variants of key driver mutations for colorectal cancer: APC, KRAS, BRAF, PIK3CA, SMAD4 and p53 is considered. This variant data will be available in the nucleosomal sequencing data.

SA1: A curated set of 12 MNase-sensitivity-derived qPCR primer sets from an initial set of 48 is developed. These provide the foundation for the development of the qPCR assay in SA2.

### Example 6. SA 2

Validate diagnostic biomarker primer sets in clinical specimens. Diagnostic biomarker primer sets are validated in a study using 96 cancer-free and 96 cancer-adjacent archived patient-derived benign polyps. An initial process to dissociate polyp tissue and isolate DNA for qPCR analysis is developed. The performance, sensitivity and specificity of each diagnostic primer set is determined. A specificity and sensitivity of at least 95% for the qPCR test for each of one control and three diagnostic primers is obtained.

50 mg of samples from SA1 is used to develop a reliable 3-5 step protocol for the processing of clinical polyp samples for MNase-sensitivity qPCR. A streamlined qPCR protocol for SARS-CoV-2 detection 3 was developed previously. Briefly, polyp samples are resuspended in MNase Buffer containing 1% formaldehyde and crosslinked at room temperature for 10 minutes. Crosslinking is quenched with an excess of glycine and homogenized. MNase is added to 200 U/ml, and incubated at room temperature for 10 minutes. PCR enhancers, including non-ionic detergents, trehalose, and carnitine will be added to the sample, immediately followed by boiling the sample for 10 minutes. The sample is filtered and 5 μl extract will be used in a 20 μl qPCR. The four control and eight diagnostic primers identified in SA1 is tested on clinical material produced. 96 CFP and 96 CAP select primers are tested with >95% sensitivity and specificity.

Several iterations of the brief MNase sensitivity protocol detailed above are completed. Variations in MNase concentration, PCR enhancer cocktail composition, and incubation times, for example, are assayed for optimum qPCR performance. An extensive literature on streamlined sample preparation for qPCR analysis of forensic and other specimens helps guide optimization. PCR primers are redesigned if they do not meet sensitivity and specificity requirements. Given the breadth of sampling in SA1, replacement qPCR probes are identified and tested.

A robust set of one control and three diagnostic primers that distinguish CFP from CAP with a sensitivity and specificity >95% is obtained.

PSA 3 Prototype assay development. The assay platform for the streamlined preparation and testing of polyp tissue for qPCR analysis in a clinical or CLIA setting is optimized. PSA 4 Deployment of the chromatin sensitivity CRC qPCR test. A commercial kit technology is developed.

TABLE 1
PRIMER PAIRS/SEQUENCES

| | | | | SEQ |
|---|---|---|---|---|
| Primer | | | | ID |
| Name | Sequence | CAP | CFP | NO: |
| MINK1_ | CCCTCTGCTC | sensitive | resistant | 1 |
| C2DeDif_ | CCTTCTCTCT | | | |
| F | | | | |
| MINK1_ | AAAGGTTCTC | | | 2 |
| C2DeDif_ | CAGGTGATGG | | | |
| R | | | | |
| PPP2R3A_ | GGCCCTCCCA | sensitive | resistant | 3 |
| C2DeDif_ | CTTTTCAG | | | |
| F | | | | |
| PPP2R3A_ | GGAAGGAAAA | | | 4 |
| C2DeDif_ | GAAAGCCTCA | | | |
| R | | | | |
| MS4A5_ | GCCTGAGAGA | sensitive | resistant | 5 |
| C2DeDif_ | TTTTGCGTTC | | | |
| F | | | | |
| MS4A5_ | GAGCTGAAAT | | | 6 |
| C2DeDif_ | TGCACCACTG | | | |
| R | | | | |
| BPNT1_ | GCTCTCCGTA | resistant | sensitive | 7 |
| C2DeDif_ | GGTGCAAGTC | | | |
| F | | | | |
| BPNT1_ | GCTGGAAGAC | | | 8 |
| C2DeDif_ | AGCTGAAACC | | | |
| R | | | | |
| LINC_ | CCCTTTCTGG | resistant | sensitive | 9 |
| C2DeDif_ | CCATAGTGAC | | | |
| F | | | | |
| LINC_ | AATGCAGAAG | | | 10 |
| C2DeDif_ | GCAAGTCTCC | | | |
| R | | | | |
| MARCHF2_ | AACTGCTAAG | resistant | sensitive | 11 |
| C2DeDif_ | TGGCCAATGC | | | |
| F | | | | |
| MARCHF2_ | TTGACTACCC | | | 12 |
| C2DeDif_ | CGTTTTGAGC | | | |
| R | | | | |
| COG2_ | CTGTGGGGAC | | | 13 |
| C2DeC_ | AGAGGCTAAG | | | |
| F | | | | |
| COG2_ | CCCTGAGACT | | | 14 |
| C2DeC_ | GCTCTGCTTC | | | |
| R | | | | |
| SUPT4H1_ | GGCTCCAACG | | | 15 |
| C2DeC_ | TAAGTTCACC | | | |
| F | | | | |
| SUPT4H1_ | GGGCTGTGGG | | | 16 |
| C2DeC_ | ACAGTAAAGA | | | |
| F | | | | |
| AP1G2_ | TTTCCTGCAA | sensitive | resistant | 17 |
| C2CuDif_ | GACTTGGTGT | | | |
| F | TG | | | |
| AP1G2_ | CCCACCACAC | | | 18 |
| C2CuDif_ | ATACCCTCTT | | | |
| R | C | | | |
| HSD3B7_ | TCACACTGTG | sensitive | resistant | 19 |
| C2CuDif_ | GGATGAGGTC | | | |
| F | | | | |
| HSD3B7_ | CCCATTCCAG | | | 20 |
| C2CuDif_ | GAACAGAGAC | | | |
| R | | | | |
| NYNRIN_ | GGCTCCCCAC | sensitive | resistant | 21 |
| C2CuDif_ | CCTACTCTAC | | | |
| F | | | | |
| NYNRIN_ | TCCCCTCTTG | | | 22 |
| C2CuDif_ | CTAGTGGAAG | | | |
| R | | | | |
| PRR7_ | GGAGGAGAAT | resistant | sensitive | 23 |
| C2CuDif_ | GGCCTCTGAC | | | |
| F | | | | |
| PRR7_ | GCCAGTCACC | | | 24 |
| C2CuDif_ | TCTAGGATGC | | | |
| R | | | | |
| SATB1_ | CCCTCTACTT | resistant | sensitive | 25 |
| C2CuDif_ | CCTGCACACC | | | |
| F | | | | |
| SATB1_ | GAGCCTGCTT | | | 26 |
| C2CuDif_ | ATTGTTGCAC | | | |
| R | | | | |
| SLC13A2_ | CAGCCTCCAA | resistant | sensitive | 27 |
| C2CuDif_ | GAACAGAAGG | | | |
| F | | | | |
| SLC13A2_ | ACCCAGTCTG | | | 28 |
| C2CuDif_ | GCAATCAGAG | | | |
| R | | | | |
| SLC35D2_ | CGGCAGGAAC | resistant | sensitive | 29 |
| C2CuDif_ | AGGAAGAG | | | |
| F | | | | |
| SLC35D2_ | AAGCAAAACA | | | 30 |
| C2CuDif_ | AGCCCTGAGA | | | |
| R | C | | | |
| UBOX5_ | CTCAAGCTGG | sensitive | resistant | 31 |
| C2CuDif_ | AGAGCTACGG | | | |
| F | | | | |
| UBOX5_ | GGAAGAGAGG | | | 32 |
| C2CuDif_ | TTGGCATCAG | | | |
| R | | | | |
| CCDC144A_ | GCTGCAGATG | | | 33 |
| C2CuC_ | CAGTGAAGTC | | | |
| F | | | | |
| CCDC144A_ | GATCACTGCC | | | 34 |
| C2CuC_ | ACGCCAAG | | | |
| R | | | | |
| PTCHD3_ | CAAGGAACGC | | | 35 |
| C2CuC_ | TTCTTCAAGG | | | |
| F | | | | |
| PTCHD3_ | CTGGGTGAAT | | | 36 |
| C2CuC_ | GCCAAGAAAG | | | |
| R | | | | |
| MIR4314_ | AGTGAGAAAG | | | 37 |
| C2CuC_ | CTGGCCTGTC | | | |
| F | | | | |
| MIR4314_ | CAGATCCCCT | | | 38 |
| C2CuC_ | TCTCCTACCC | | | |
| R | | | | |
| STAG2_ | GACCGCCACT | | | 39 |
| C2CuC_ | TTCAAAACC | | | |
| F | | | | |
| STAG2_ | TACACAGAGG | | | 40 |
| C2CuC_ | CAGCAACGAG | | | |
| R | | | | |
| RXFP3_ | GCCACCGTAA | | | 41 |
| C1CuDif_ | CTGGTGAGAG | | | |
| F | | | | |
| RXFP3_ | GTTTCCTCTG | | | 42 |
| C1CuDif_ | GGACTGCTTG | | | |
| R | | | | |
| TIPARP_ | AGGAGGCTGG | | | 43 |
| C1CuDif_ | ACAGGAGTTG | | | |
| F | | | | |
| TIPARP_ | AGTGACGCCT | | | 44 |
| C1CuDif_ | CCTGTTGG | | | |
| R | | | | |
| HAUS4_ | GACTGGGAAA | | | 45 |
| C1CuDif_ | CAGGATGGTC | | | |
| F | | | | |
| HAUS4_ | GCAAAATGCT | | | 46 |
| C1CuDif_ | TGGCGTAGTG | | | |
| R | | | | |

What is claimed is:

1. A method to determine differentiation potential of a cell, the method comprising:
a) exposing a sample comprising nucleosomes to Micrococcal nuclease (MNase) under conditions where MNase can digest nucleic acid, thereby producing digested nucleic acid;
b) detecting the digested nucleic acid;
c) comparing the digested nucleic acid of the sample to a standard, and
c) determining differentiation potential of the cell.

2. The method of claim 1, wherein the differentiation potential is selected from the group comprising: cancer differentiation potential, pre-cancer differentiation potential, and/or developmental differentiation potential.

3. The method of claim 1, wherein the sample comprising nucleosomes comprises whole cells, cell components, and/or cell extracts.

4. The method of claim 1, wherein the digested nucleic acid is detected by amplification.

5. The method of claim 4, wherein the amplification is selected from the group comprising: ligase chain reaction (LCR), loop mediated isothermal amplification (LAMP), multiple displacement amplification, self-sustained sequence replication (3SR), rolling circle amplification (RCA), nucleic acid sequence-based amplification (NASBA), polymerase chain reaction (PCR), and quantitative polymerase chain reaction (qPCR).

6. The method of claim 4, wherein the amplification utilizes primer pairs comprising the nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; or primer pairs with 80% or more homology to these sequences.

7. The method of claim 1, wherein differentiation potential indicates a higher probability of pathology compared to a normal cell.

8. The method of claim 7, wherein the sample comprising nucleosomes is a pathology sample.

9. The method of claim 8, wherein the digested nucleic acid is detected by qPCR.

10. The method of claim 8, wherein the pathology sample is a colon polyp.

11. The method of claim 7, wherein the pathology potential is cancer potential and/or an inflammatory potential.

12. The method of claim 1, which has greater than 90% sensitivity and specificity.

13. The method of claim 7, which provides pathology potential determination the same day as the pathology sample is removed from a patient.

14. A method of predicting, preventing, diagnosing, or treating a disease in a subject in need thereof, the method comprising determining the differentiation potential of a cell according to claim 1, and predicting, preventing, diagnosing, or treating a disease if the pathology potential is high.

15. The method of claim 14, wherein the disease is a neurodegenerative disease, inflammatory disease, viral infection, or a cancer.

16. A method of predicting, preventing, diagnosing, or treating a disease in a subject in need thereof, the method comprising determining the differentiation potential of a cell according to claim 1, and predicting, preventing, diagnosing, or treating a disease if the pathology potential is low.

17. The method of claim 16, wherein the disease is a neurodegenerative disease, inflammatory disease, viral infection, or a cancer.

18. A kit comprising MNase and primers for amplifying nucleic acid digested by MNase.

19. The kit of claim 18, which further comprises control primers.

20. The kit of claim 18, wherein the primers are in pairs comprising nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46, or primer pairs with 80% or more homology to these primers.

21. The kit of claim 18, wherein the kit further comprises components for amplification of nucleic acid.

22. Isolated primer pairs comprising nucleic acid sequences selected from the group comprising: SEQ ID NO:1 and SEQ ID NO:2; SEQ ID NO:3 and SEQ ID NO:4; SEQ ID NO:5 and SEQ ID NO:6; SEQ ID NO:7 and SEQ ID NO:8; and SEQ ID NO:9 and SEQ ID NO:10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46, or primer pairs with 80% or more homology to these primers.
